(19) United States
(12) Patent Application Publication
(10) Pub. No.: US 2022/0160934 A1
(43) Pub. Date: May 26, 2022

(54) INJECTABLE HOMOGENEOUS GELS COMPRISING MULTIPLE FORMS OF HYALURONIC ACID AND METHODS FOR MANUFACTURING THEREOF

(71) Applicant: ALLERGAN PHARMACEUTICALS INTERNATIONAL LIMITED, Dublin 17 (IE)

(72) Inventors: David Dadi SEGAL, Tel Aviv (IL); Liat GOLDSHAID-ZMIRI, Petach Tikva (IL); Lital SHKLANOVSKY, Kiryat Ono (IL); Eran GOLDBERG, Ramat Gan (IL)

(73) Assignee: ALLERGAN PHARMACEUTICALS INTERNATIONAL LIMITED, Dublin 17 (IE)

(21) Appl. No.: 17/441,943

(22) PCT Filed: Mar. 23, 2020
(86) PCT No.: PCT/IL2020/050342

§ 371 Date: Sep. 22, 2021

Related U.S. Application Data
(60) Provisional application No. 62/822,896, filed on Mar. 24, 2019.

(51) Int. Cl. A61L 27/20

(52) U.S. Cl. CPC A61L 27/20; A61L 2430/34; A61L 2400/06; A61L 2300/802

(57) ABSTRACT

Provided herein are compositions, comprising hyaluronic acid in three different forms. The compositions are in a form of an essentially homogeneous gel with improved rheological properties enabling improved clinical performance.

### FIELD OF THE INVENTION

The present invention relates to homogeneous compositions comprising composites of processed hyaluronic acid, methods of manufacturing of such homogeneous compositions, and uses thereof in cosmetic applications, and medical and pharmaceutical applications.

### BACKGROUND OF THE INVENTION

Hyaluronic acid is a natural polysaccharide which is a common component of cosmetic preparations and is used in several cosmetic procedures, particularly as a dermal filler. However, natural hyaluronic acid has poor in-vivo stability due to rapid enzymatic degradation and hydrolysis. Various chemical modifications have been proposed, such as cross-linking, in attempt to improve the poor stability of natural hyaluronic acid.

Dehydration of the cross-linked hydrogel can sometimes be used to improve mechanical properties of the hydrogel as can be seen at patent application US 20160376382 A1. According to the document, an effective cross-linking process involves activation of the cross-linking, followed by breakdown and maturation of the gel particles under dehydrating conditions during a precipitation step, and followed by a drying of the gel. The dry gel is allowed to swell into a gel from a buffer.

In patent EP2011816A1 a highly cross-linked gel was prepared and then dried, to be further subjected to a second cross-linked process (lightly cross-linked), followed by neutralization and stabilization in phosphate buffer to produce a co-crosslinked hydrogel.

In the U.S. Pat. No. 8,450,475 sterile injectable compositions of lidocaine and cross-linked and free hyaluronic acid forms are disclosed. Particles of relatively highly cross-linked hyaluronic acid are dispersed in free hyaluronic acid solution, at various ratios, e.g. 8 parts of particles in 2 parts of solution. Further, the U.S. Pat. No. 9,358,322 discloses that the free hyaluronic acid phase may be relatively less cross-linked.

There is a need in the art to provide hyaluronic acid composite materials with improved properties, such as degradation rate, spatial swelling behavior and/or improved rheological properties, which could ultimately result in improved performance in vivo.

The present invention provides a homogeneous composition comprising dried ground powder of a first cross-linked gel, dispersed in and merged with a phase comprising a mixture of a further cross-linked gel and free hyaluronic acid.

### SUMMARY OF THE INVENTION

Provided herein are composite materials, methods of manufacture thereof, and uses thereof as cosmetic compositions, or as pharmaceutical compositions, as described in greater detail below. In one aspect, the composite materials are homogeneous compositions of free hyaluronic acid (sometimes referred to as non-cross-linked gel, or “NCL-gel”), cross-linked hyaluronic acid (sometimes referred herein as “CL-gel”), and of dried highly cross-linked hyaluronic acid (sometimes referred herein as “DHCL”). It has now been unexpectedly found that combining these three components, as generally described herein, can furnish an essentially homogeneous composition, with improved elasticity and stability, and no detectable phase separation or boundary. Usually, as described in greater detail below, the ratios between the components of the composite are chosen such that an essentially homogeneous gel is obtained when these three components are mixed together. As demonstrated in the appended examples herein below, the composite materials according to the invention increase elasticity at near-zero deformation, thereby ensuring minimal deformation and migration potential when in use. Without being bound by a particular theory it is believed that the inclusion of the DHCL into a phase comprising NCL-gel and CL-gel leads to a stabilization of the composite gel at rest, and to interactions of the swollen DHCL particles between themselves and other gel components at near-zero shear, thereby increasing the stability of the structure and thus its elasticity and resilience to initiation of flow.

Thus, in a first aspect provided herein a process of manufacturing of essentially homogeneous composite hyaluronic acid-based materials, comprising combining a free hyaluronic acid, a cross-linked hyaluronic acid, and a dried highly cross-linked hyaluronic acid, preferably in an aqueous medium. Preferably, in an arbitrary order, a solution of free hyaluronic acid is combined with a first cross-linked hyaluronic acid gel, and further combined with dried further gel of cross-linked hyaluronic acid, preferably higher cross-linked than the first gel. Preferably, the amount of the dried highly cross-linked hyaluronic acid is between 0.2 weight percent and 1.5 weight percent.

In a further aspect provided herein an essentially homogeneous gel comprising free hyaluronic acid, cross-linked hyaluronic acid, and dense cross-linked hyaluronic acid. The cross-linked hyaluronic acid is usually a gel in water or an essentially aqueous medium, which can be referred to as “structure gel”. The dense cross-linked hyaluronic acid may usually be in form of at least partially swollen dried powder of cross-linked hyaluronic acid gel, which can be referred to as “precursor gel”, to differentiate from the “structure gel”. The degree of cross-linking is usually higher in the precursor gel than in the structure gel. The essentially homogeneous gel may further comprise a local anesthetic. Preferably, the essentially homogeneous gel is a gel prepared by steps comprising combining cross-linked hyaluronic acid gel with dried highly cross-linked hyaluronic acid and with free hyaluronic acid solution. In a further aspect provided herein use of an essentially homogeneous gel as described herein, in cosmetic procedures, e.g. wrinkle filling, or in a medical or pharmaceutical application.

Generally, in a first aspect, provided herein is a process of manufacturing a hyaluronic acid composition, said process comprises combining free hyaluronic acid gel, cross-linked hyaluronic acid gel, and dried highly cross-linked hyaluronic acid gel, and mixing to obtain an essentially homogeneous gel which is homogeneous upon visual inspection versus ample light source and under magnification of up to ×3. In the process said free hyaluronic acid gel may be provided by combining hyaluronic acid and an aqueous buffer solution, and mixing until dissolution. In the process said cross-linked hyaluronic acid gel may be provided by combining in an aqueous medium hyaluronic acid or a salt thereof and a cross-linking agent, subjecting the resultant mixture to cross-linking conditions, and completing the cross-linking reaction. The subjecting to cross-linking conditions may comprise increasing the pH of the medium, and said completing the cross-linking reaction may comprise allowing the reaction mixture to stand, and/or neutralizing said reaction mixture. The dried highly cross-linked hyaluronic acid may be provided by drying a precursor gel of highly cross-linked hyaluronic acid, and grinding it to particle size below 500 microns, preferably to below 250 microns. The drying may be effected by lyophilizing. The precursor gel of highly cross-linked hyaluronic acid may be provided by combining in an aqueous medium hyaluronic acid or a salt thereof and a cross-linking agent, subjecting the resultant mixture to cross-linking conditions, and completing the cross-linking reaction, and preferably the subjecting to cross-linking conditions may comprise increasing the pH of the medium, and said completing the cross-linking reaction may comprise allowing the reaction mixture to stand, and/or neutralizing said reaction mixture. In the process, the cross-linking agent may be 1,4-butanediol diglicydyl ether (BDDE). The amount of said cross-linking agent in said precursor gel of highly cross-linked hyaluronic acid may be between 150% and 500% higher than corresponding amount of said cross-linking agent in said cross-linked hyaluronic acid gel, on weight basis relative to a respective amount of hyaluronic acid. The amount of said free hyaluronic acid gel may be between 5 and 45 weight percent of the total weight of said essentially homogeneous gel, optionally between 7 and 25 weight percent. The an amount of said dried highly cross-linked hyaluronic acid gel may be between 0.25 and 3.5 weight percent of the total weight of said essentially homogeneous gel. The amount of said cross-linked hyaluronic acid gel may be between 45 and 95 weight percent of the total weight of said essentially homogeneous gel, optionally between 70 and 95 weight percent. The process may further comprise sterilizing said essentially homogeneous gel, optionally by autoclaving said essentially homogeneous gel. The homogeneous gel produced according to the process may be inseparable by centrifugation up to 120 minutes at 16,000 g-force. The homogeneous gel according to the process may further be characterized in that that a plot of viscous modulus G″ versus frequency demonstrates a local minimum in near-zero region at frequencies between 0 and 5×10⁻³ Hz, at frequency sweep test of said homogeneous composition.

In an additional aspect there is provided a hyaluronic acid composition comprising water, free hyaluronic acid, cross-linked hyaluronic acid, and dense highly cross-linked hyaluronic acid, wherein said composition comprises between 0.5 and 9 weight percent of hyaluronic acid, wherein said composition is injectable, wherein said composition is an essentially homogeneous gel upon visual inspection versus ample light source and under magnification of up to ×3. In the composition said dense highly cross-linked hyaluronic acid may be at least partially swollen particle of dried highly cross-linked hyaluronic acid gel. The cross-linked hyaluronic acid and the dense highly cross-linked hyaluronic acid comprise hyaluronic acid cross-linked with a cross-linking agent, wherein an amount of said cross-linking agent in said dense highly cross-linked hyaluronic acid is between 150% and 500% higher than the amount in said cross-linked hyaluronic acid, on weight basis relative to a respective amount of hyaluronic acid. In the composition the cross-linking agent may be 1,4-butanediol diglicydyl ether (BDDE). In the composition the amount of said free hyaluronic acid may be between 5 and 45 weight percent of the total weight of said composition, optionally between 7 and 25 weight percent. In the composition the amount of said cross-linked hyaluronic acid is between 45 and 95 weight percent of the total weight of said composition, optionally between 70 and 95 weight percent. In the composition the amount of said dense highly cross-linked hyaluronic acid is between 0.25 weight percent and 3.5 weight percent. The composition may be inseparable by centrifugation for up to 120 minutes at 16,000 g-force. The composition may further be characterized in that that a plot of viscous modulus G″ versus frequency demonstrates a local minimum in near-zero region at frequencies between 0 and 5×10−3 Hz, at frequency sweep test of said homogeneous composition.

### BRIEF DESCRIPTION OF THE FIGURES

FIG. 1 demonstrates a rheogram obtained from frequency sweep measurement of a composition according to the invention comprising 0.5 weight percent of DHCL. In the graph, hollow triangles (Δ) represent elastic modulus G′, hollow squares (□) represent viscous modulus G″, and the hollow circles (∘) represent the phase angle δ.

FIG. 2 demonstrates a rheogram obtained from frequency sweep measurement of a composition according to the invention comprising 1 weight percent of DHCL. In the graph, hollow triangles (Δ) represent elastic modulus G′, hollow squares (□) represent viscous modulus G″, and the hollow circles (∘) represent the phase angle δ.

FIG. 3 demonstrates a rheogram obtained from frequency sweep measurement of a comparative composition comprising CL-gel only. In the graph, hollow triangles (Δ) represent elastic modulus G′, hollow squares (□) represent viscous modulus G″, and the hollow circles (∘) represent the phase angle δ.

### DETAILED DESCRIPTION OF THE INVENTION

The homogeneous gel according to the invention is usually formed when three structural components (i.e. CL-gel, NCL-gel, and DHCL, provided as cross-linked hyaluronic acid, free hyaluronic acid, and dense highly cross-linked hyaluronic acid) are combined to furnish an essentially uniform structure. It is believed, without being bound by any particular theory, that the dried cross-linked hyaluronic acid particles swell at least to a certain extent upon contact with the other components, particularly with the non-cross-linked hyaluronic acid solution, yet their swelling may be restricted by the presence of the cross-linked hyaluronic acid gel. These at least partially swollen particles may interact with the cross-linked gel, e.g. via the cohesion forces, thus forming an essentially uniform phase, with swollen particles being uniformly distributed throughout the bulk of the cross-linked gel. The process may be assisted by the presence of free hyaluronic acid, thereby increasing the cohesion. When sheared, it is believed that the interaction forces between the at least partially swollen particles and between the particles and the cross-linked gel increase the impedance to flow.

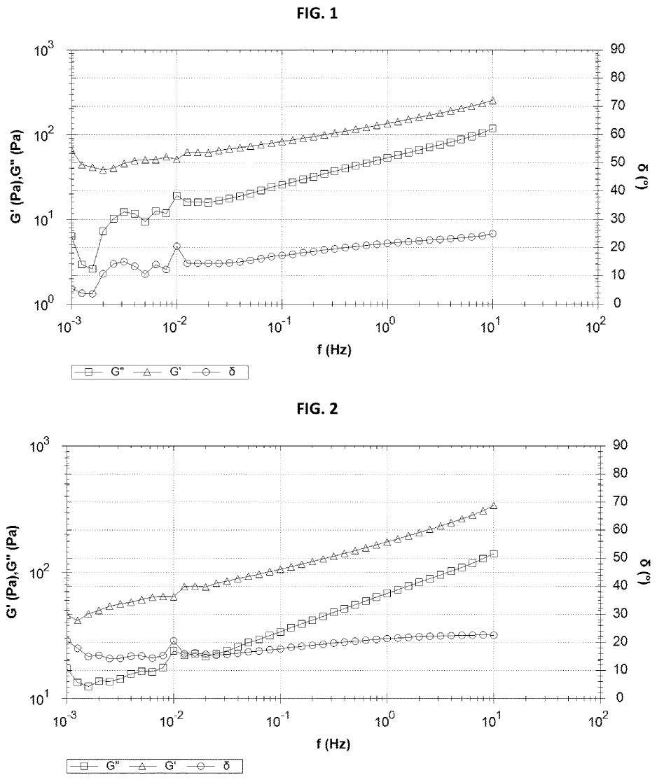

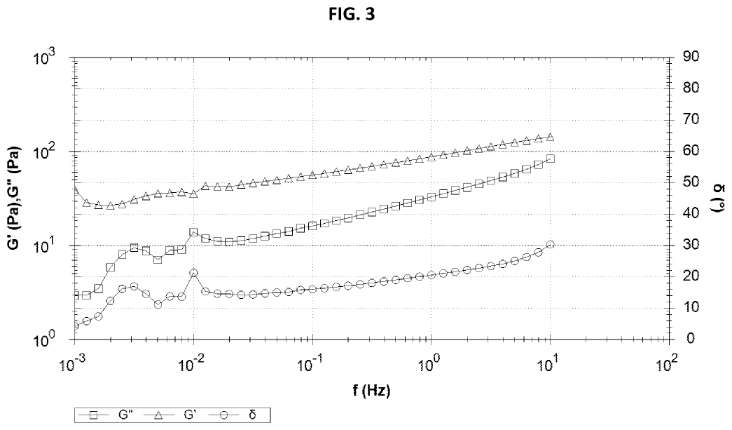

This increased impedance may produce dilatant-like rheological behavior at near-zero shear, which can be detected in the essentially homogeneous gel, as demonstrated in the appended Examples, e.g. by oscillatory rheometry testing, and identifying at least some decrease in viscous component (G″) of the composite modulus G*, which may also be paralleled by a decrease in the angle δ and consequently in tan δ, indicating the increase in overall elasticity of the viscoelastic composition. This change in the G″ viscous modulus may usually be observed at frequency sweep test, e.g. at shearing strain of about 0.5%, in a plot of viscous modulus G″ versus frequency, where it demonstrates a local minimum in near-zero region. The local minimum does not occur at the limit values of the tested range, but rather is usually observed after an initial decrease starting from the lowest tested frequency towards higher frequencies, particularly between 1×10⁻³ Hz and 5×10⁻³ Hz. Generally, the near-zero region may thus be viewed as frequencies range from 0 to about 5×10⁻³ Hz, depending on the capabilities of the tested equipment, and it is in this range, i.e. upon initiation of deformation/flow, that the homogeneous gel of the present invention demonstrates the decrease in viscous component, as can be seen, for example, in the FIGS. 1 and 2, but not in FIG. 3, which is a rheogram of a comparative example.

This decrease in viscous component of the composition and thus increased resistance to flow initiation is a very beneficial property for a composition that may be used in cosmetic applications, such as tissue filling, as the composition thus remains at the site of application and does not migrate spontaneously, the factor that in the existing tissue filling solutions deprecate the treatment efficiency and create concerns about side effects.

Thus, the gel according to the invention is essentially homogeneous. This homogeneity is firstly manifested in that that the gel is visually uniform, and is preferably clear. Upon visual inspection in a transparent container versus ample light source a clear gel with no visually perceptible structure or ordered particulate matter can be seen, at least up to magnification of ×3. Moreover, as demonstrated in the appended examples below, the gel could not be separated by centrifugation at 16,000 g for 120 minutes. Generally, the essentially uniform gel has all the structural components uniformly distributed throughout the bulk, e.g. in a final transparent container, and cannot be visually discerned, at least at magnification of up to ×3. The term homogeneous thus does not necessarily imply complete homogeneity on molecular level, rather the uniformity of distribution of the structural components of the gel, e.g. DHCL, NCL-gel and CL-gel, and preferably a certain degree of cohesion therebetween to create an essentially uniform composition.

The main component of the compositions according of the invention is hyaluronic acid, which is present in several chemically different forms. The first form is free hyaluronic acid, i.e. hyaluronic acid that was not chemically modified to form links with other molecules, particularly with other hyaluronic acid molecules. The further forms are cross-linked hyaluronic acid, that is different in a degree of cross-linking and/or further processing, such as drying. The total concentration of hyaluronic acid, i.e. of free hyaluronic acid, cross-linked hyaluronic acid, and dried highly cross-linked hyaluronic acid, in the composition, may vary from 0.1 weight percent to 9 weight percent, e.g. from 0.1 weight percent to 4 weight percent, or from 0.5 weight percent to 9 weight percent, preferably between 0.5 weight percent and 4 weight percent, dependent on many factors, e.g. the molecular weight of hyaluronic acid. In some embodiments, e.g. when the molecular weight of hyaluronic acid is about 0.8-3.5 MDa, the concentration of hyaluronic acid is preferably between 0.8 and 3.5 weight percent (values' similarity is coincidental), e.g. between 2 and 3.3 weight percent, or between 2.2 and 3.3 weight percent. Apart from other advantages of the compositions of the present invention, as demonstrated in the appended examples, the concentrations of hyaluronic acid attainable in an injectable gel by providing hyaluronic acid as free hyaluronic acid, cross-linked hyaluronic acid, and dense highly cross-linked hyaluronic acid, is higher that could be obtained with just plain cross-linked hyaluronic acid gel, at similar degree of the cross-linking, viscosity and/or injection force. In other words, preparing a gel comprising the same amounts of hyaluronic acid and the cross-linking agent could result in a gel that cannot be readily handled or used, due to high viscosity and/or very high injection force; to accommodate the increased amount of hyaluronic acid, modifications could be necessary, either a decrease in cross-linking density, e.g. through less cross-linking agent or less efficient cross-linking conditions, a significant decrease in molecular weight of hyaluronic acid, or other similar changes, which could inevitably have bearing on in-vivo performance of the gel.

The homogeneous gels according to the invention are readily injectable, e.g. no excessive force is required to inject the gel through a needle at common injection rate. For example, the gels may be injectable through a regular medical or cosmetic needle, e.g. 25G/16-mm needle. The injection rate may be from 0.2 mL per minute to 1.5 mL per minute, preferably between 0.9 mL/min and 1.1 mL/min. The force required to inject the gels may vary according to their respective composition and the concentration of hyaluronic acid components, but generally when extruded through the 25G needle the average force required to force the gel from a standard 1-mL syringe with 6.35±0.1 mm inner diameter is less than 40 Newton.

In the context of the present invention, the terms “hyaluronic acid”, “HA” or “hyaluronate” refer interchangeably to a linear polysaccharide or to its salt, particularly to a nonsulfated glycosaminoglycan, composed of a repeated disaccharide units, each unit consisting of D-glucoronic acid, or its salt, and D-N-acetylglucosamine, via alternating β-1,4 and β-1,3 glycosidic bonds. Hyaluronic acid or salts thereof may come from a variety of sources in a variety of molecular weights and other specifications. Generally, all sources of hyaluronic acid may be useful for the purposes of the present invention, including bacterial and avian sources.

The molecular weight of hyaluronic acid may be used in order to describe the material. The term “molecular weight” may refer to both the weight-average molecular weight and the number-average molecular weight, as known in the field of polymers. Useful hyaluronic acid materials may have a molecular weight of from about 0.25 MDa (mega Dalton) to about 4.0 MDa, preferably between about 0.8 MDa to about 3.5 MDa. Hyaluronic acid may be further characterized with a polydispersity value of its molecular weight, indicative of the variation of the molecular weights in the polymer. While it may be advantageous to use a low-polydispersity hyaluronic acid for the sake of improved repeatability of the processes, it may be economically infeasible. A reasonable compromise between the width of the molecular weights polydispersity and the price of the starting material may be achieved, and suitable hyaluronic acid materials may preferably have a polydispersity from about 1.1 to 4.0, preferably less than 3.0, further preferably less than 2.0.

Generally, the cross-linked hyaluronic acid in the composition is hyaluronic acid that was combined with a cross-linking agent at cross-linking conditions, as described herein. Thus, cross-linked hyaluronic acid is a plurality of hyaluronic acid molecules chemically bound to divalent cross-linker molecules' residues, forming thereby interconnected network of said hyaluronic acid molecules. The size and the density of the network is usually controlled by the amount of cross-linking residues bound to the hyaluronic acid molecules, which is referred to herein as a degree of cross-linking.

The term “cross-linking agent”, “cross-linker” and the like, as used interchangeably herein, refer to molecules that contain at least two reactive functional groups that create covalent bonds between two or more molecules of hyaluronic acid. The cross-linking agents can be homo-bi functional (i.e. have two reactive ends that are identical) or hetero-bifunctional (i.e. have two different reactive ends). The terms “cross-linker residues” or “cross-linking molecules' residues” and the like, as used interchangeably herein, refer to the groups creating the covalent bonds between the hyaluronic acid molecules, the groups being an adduct reaction product of the cross-linking agent and hyaluronic acid. The cross-linking agents suitable for use in the present invention usually comprise complementary functional groups to that of hyaluronic acid such that the cross-links could be formed. Preferably, the cross-linking does not form esterified hyaluronic acid. Non-limiting examples of cross-linking agents suitable for the present invention include 1,4-butanediol diglycidyl ether (BDDE), 1,2,7,8-diepoxyoctane (DEO), biscarbodiimide (BCDI), adipic dihydrazide (ADH), bis-(sulfosuccinimidyl)-suberate (BS3), hexamethylenediamine (NMDA), 1-(2,3-epoxypropyl)-2,3-epoxycyclohexane, multifunctional cross-linking agents such as pentaerythritol tetraglycidyl ether (PETGE) or PEG based such as polyethylene diglycidyl ether (PEGDE), mono ethylene glycol diglycidyl ether (EGDE), or a combination thereof. The same or different cross-linker may be used for different components of the composition, e.g. for DHCL and CL-gel. Preferably, the cross-linking agent is BDDE, for both components.

The composite gel according to the invention comprises dried highly cross-linked hyaluronic acid (DHCL), which is at least partially swollen in presence of the further components of the gel. The DHCL is provided as particulate matter, preferably a powder with particle size between 25 microns (i.e. micrometers) and 500 microns, e.g. 50 to 300 microns, further preferably in one or more of the following particle size ranges: 45 microns to 105 microns, 95 microns to 155 microns, 145 microns to 255 microns, and/or 245 microns to 410 microns, or mixtures thereof. Preferably, the particle size of the DHCL powder is between 50 and 250 microns. The exact particle size range boundaries will be determined by the method of manufacture of the particles and the particle size determination methods, such that all the values within about 10% of the stated values that are represented by the specific values presented herein. It is readily appreciable that DHCL particles may readily change dimensions when introduced into the composite essentially homogeneous gels of the invention, as they at least partially swell in presence of the other components. The particles may even transform into merely gel areas with increased density relatively to the remainder of the bulk. Thus, in the final gels, the dimensions of the dense gel areas may be significantly larger than the original DHCL particles. The boundaries of these areas, however, may not be readily discernable. As to the density of these denser areas in the final gel, these may be significantly increased due to interaction with other components of the gel, e.g. due to some restriction to the swelling of the particles.

The DHCL particles, and consequently the at least partially swollen DHCL particles, comprise cross-linked hyaluronic acid. The degree of cross-linking in DHCL is high, relative to cross-linked hyaluronic acid (CL-gel). Generally, the degree of cross-linking, expressed in weight ratio between the amount of cross-linker and the cross-linked hyaluronic acid, is between 150% and 500%, preferably between two to three times higher that of CL-gel. The degree of cross-linking may be dependent on the molecular weight and the nature of the cross-linker. The preferred cross-linker for DHCL is 1,4-butanediol diglycidyl ether (BDDE). Particularly, when BDDE is the cross-linking agent and hyaluronic acid has a molecular weight of between 0.8-3.5 MDa, the preferred degree of cross-linking in DHCL may be between 12 to 30 percent, by weight of the cross-linker to total weight of cross-linked hyaluronic acid, such as between 19 and 25 percent.

The amount of DHCL in the essentially homogeneous gel composition is usually between 0.25 and 4 weight percent, preferably between 0.3 and 1.5 weight percent, further preferably between 0.35 and 0.65 weight percent, or between 0.8 and 1.2 weight percent.

Solid DHCL particles may usually be produced by drying a precursor hyaluronic acid gel. The precursor gel is usually highly cross-linked hyaluronic acid gel in an aqueous medium, such as water or an aqueous buffer. The precursor gel could be ground and dried, to furnish DHCL particles, as discussed in greater detail below. Drying of hyaluronic acid gels was described in the art by some methods, mostly by dehydration of ground gels in organic solvents, such as low alcohols. However, preferably the drying of the precursor gel is performed by lyophilization, followed by grinding the lyophilizate to desired particle size. Without being bound by any particular theory it is believed that particles produced in this way undergo cohesion into the homogeneous gel more readily than particles obtained by first grinding of a gel and then dehydrating it, presumably due to significantly reduced interfacial effects that may be associated with dehydration in organic solvents. The precursor gel may comprise cross-linked hyaluronic acid in a final concentration of between 0.5 and 8 weight percent, preferably between 2.5 and 4.9 weight percent. In some further embodiments, however, the concentration of hyaluronic acid in the precursor gel may be between 0.1 and 3.5 weight percent, preferably between 0.5 and 3.5 weight percent. The precursor gel may further comprise salts, such as buffers, e.g. that were used during the cross-linking and/or neutralization stage, lyophilization-assisting additives, and other excipients.

The compositions of the invention also comprise cross-linked hyaluronic acid gel component, i.e. the structure gel. The structure gel may usually comprise hyaluronic acid in a concentration between 0.5 and 5 weight percent, e.g. between 0.7 and 3 weight percent, preferably between 0.8 and 2.4 weight percent. Hyaluronic acid used for the CL-gel component may be the same or different from what is used for the DHCL component, along the general lines described above. The cross-linking ratio, on the other hand, is significantly lower than for the DHCL component. As in the case of DHCL, the degree of cross-linking may be dependent on the molecular weight and the nature of the cross-linker. The preferred cross-linker for CL-gel component is also 1,4-butanediol diglycidyl ether (BDDE). Particularly, when BDDE is the cross-linking agent and hyaluronic acid has a molecular weight of between 0.8-3.5 MDa, the preferred degree of cross-linking in CL-gel may be between 7 to 20 percent, by weight of the cross-linker to total weight of cross-linked hyaluronic acid, such as between 7.5 and 10 percent, between 10 and 15 percent, or between 15 and 17 percent. The CL-gel component may further comprise salts, such as buffers, e.g. that were used during the cross-linking and/or neutralization stage, and other excipients.

As the CL-gel usually provides the main structure to the essentially homogeneous compositions of the invention, it is usually present as a majority component, e.g. between 45 and 95 weight percent of the composition, preferably equal to or above 50 percent by weight, excluding the DHCL component, for simplification of the calculation and for demonstration purpose. More preferably, the amount of CL-gel is between 60 and 90 weight percent of the composition, excluding the DHCL component. Further preferably, the CL-gel component is present in an amount between 75 and 90 weight percent of the total composition.

The compositions of the invention also comprise non-cross-linked hyaluronic acid gel component, e.g. the free hyaluronic acid. The NCL-gel usually comprise hyaluronic acid in a concentration between 0.4 and 5 weight percent, e.g. between 0.5 and 4 weight percent, or between 1 and 3.5 weight percent, preferably between 0.7 and 3.0 weight percent, e.g. between 0.8 and 1.2 weight percent, or between 1.8 and 2.2 weight percent. Hyaluronic acid used for the CL-gel component may be the same or different from what is used for the DHCL component and for the CL-gel component, along the general lines described above. The NCL-gel component may further comprise salts, such as buffers and osmolarity adjusting agents, and other excipients.

As the NCL-gel may assist in incorporation of DHCL and/or in improving the flow properties of the composition, such as injectability (e.g. injection force), it is usually present as a minority component, e.g. equal to or less than percent by weight, excluding the DHCL component (as explained above). Preferably, the amount of NCL-gel is between 5 and 45 weight percent of the composition, excluding the DHCL component. Further preferably, the NCL-gel component is present in an amount between 7 and 25 weight percent of the total composition.

Hyaluronic acid used for each of the three components may have same or different characteristics. For example, the molecular weight of hyaluronic acid used in dried highly cross-linked component may be low and highly polydisperse, and the useful properties could be controlled by a higher degree of cross-linking. Similarly, the molecular weight of the non-cross-linked gel component may be relatively high and uniform, e.g. to enable the use of lower concentrations thereof in the solution. Preferably, however, hyaluronic acid in all the components is of comparable or identical molecular weight distribution.

The compositions of the present invention comprise water. Water is the preferable solvent for the components of the essentially homogeneous gel. Water may be pure water, but may preferably comprise inorganic salts. These salts may serve to control the pH of the composition, both of the final composition and in preparation, e.g. during a cross-linking step, as discussed in greater detail below. The salts may be used to affect the osmolarity of the gel as well. The salts present in water in the compositions according to the invention include pharmaceutically acceptable salts of alkali metals, e.g. sodium and/or potassium, and inorganic acids, e.g. a phosphoric acid, hydrochloric acid, or organic acid, e.g. citric acid, tartaric acid and the like. Preferably, buffering agents and osmolarity agents comprise sodium chloride, phosphate salts, e.g. monobasic, dibasic or tribasic salts of ortho-phosphoric acid with sodium and/or potassium. Further, osmolarity agents may include neutral hydrophilic organic compounds, such as sugars, e.g. mannitol, dextrose, and the like.

The compositions of the present invention may further comprise biologically active material, e.g. drugs. The non-limiting examples of drugs suitable for the composite gels include local anesthetic, e.g. lidocaine, prilocaine, and the like, and may also include drugs like hormones, growth factors, and steroids. In some embodiments, the compositions may further comprise inorganic particles, such as calcium hydroxyapatite.

Generally, different ratios of the three components may be present in the compositions according to the invention:
Cross-linked gel component in concentrations between 45 and 95% wt, preferably between 75 to 95% wt;
Non-Cross-linked gel component in concentrations between 5 and 45% wt, preferably between 7 to 25% wt;
Dry cross-linked gel component in concentrations between 0.25 and 4% wt, preferably between 0.3 to 1.3% wt; with the total making up to 100 percent.

The cross-linking of the hyaluronic acid components may be carried out as known in the art, e.g. as generally described in PCT patent application WO2018047182, incorporated herein by reference. Briefly, hyaluronic acid in desired amount may be dissolved in water, together with the required amount of cross-linking agent, and subjected to cross-linking conditions. The “cross-linking conditions” refer to reaction conditions that allow formation of covalent bonds between HA chains. Generally, cross-linking conditions effect the cross-linking reaction, and may include adjustment of the mixture to a desired pH and temperature, specific for a cross-linking agent used. The cross-linking conditions may include elevating the pH of the mixture to a pH above 12. The cross-linking conditions may further include exposing the mixture to elevated temperature, e.g. to 40° C.-50° C., e.g. 45° C., for a first period, e.g. between 1 and 5 hours, e.g. 3 hours. The cross-linking conditions may further include exposing the mixture to about 25° C. for a second period, e.g. 12-20 hours, preferably about 15 hours. The optimal cross-linking temperature and pH may be readily determined experimentally by testing the cross-linking conditions for HA that are well known in the art for a specific cross-linking agent. Sometimes, to terminate the cross-linking reaction the cross-linking conditions may be removed. The termination of the cross-linking reaction may include adjustment of the mixture to a desired pH and temperature, specific for a cross-linking agent used, e.g. by adjusting the pH of the mixture to a pH of about 7.

In conducting a process of manufacturing of compositions according to the invention, hyaluronic acid or a salt thereof may be added to water and mixed in a suitable mixer until dissolution. Cross-liking agent, e.g. BDDE, may be added to the mixer, and mixed until dissolution. Alternatively, a solution of cross-linking agent may be added to the solution of hyaluronic acid. Hyaluronic acid may be dispersed in the water, e.g. using a rotor-stator homogenizer, to facilitate dissolution. The conducting a cross-linking reaction may comprise increasing the pH of the medium. This may be achieved by adding to the reaction mixture a sufficient amount of a base or a solution of a base, and mixing until homogeneous. The temperature of the reaction mixture may be elevated if needed. Completing the cross-linking reaction to obtain a gel may include neutralizing said reaction mixture, i.e. to achieve a pH of about between 6.0 and 7.8, e.g. about 7, e.g. by adding an aqueous acid or a neutral or acidic buffer, or allowing the reaction to proceed to an essentially full conversion of the cross-linking agent, in which case the final pH of the mixture could be adjusted upon completion of the reaction.

The pH and osmolarity of the components may further be adjusted to physiological values, either individually or in a finished product of an essentially homogeneous gel; preferably before the combining of the components. Neutralization may be carried out by addition of aqueous solutions comprising pharmaceutically acceptable acids, buffering agents, e.g. phosphate salts and/or phosphoric acid and/or hydrochloric acid, of pH between 5 and 8, according to the requirement of the final pH, preferably to a final pH of about 7. Similarly, osmolarity adjustment may be performed by adding to the mixture a solution of salts, e.g. sodium chloride, additional phosphates as described herein, and mixing the component mixture to homogeneity.

Thus, in a further aspect provided herein a process of manufacturing of the composite essentially homogeneous gels comprising free hyaluronic acid, cross-linked hyaluronic acid, and dense highly cross-linked hyaluronic acid. The process comprises combining free hyaluronic acid gel, cross-linked hyaluronic acid gel, and dried highly cross-linked hyaluronic acid powder, in a suitable vessel, and mixing at suitable temperature, until a homogeneous gel is obtained. A free hyaluronic acid gel may be prepared by combining hyaluronic acid powder with water or buffered aqueous solution at desired pH, and mixing until dissolution. A cross-linked hyaluronic acid may be prepared by combining hyaluronic acid and a cross-linking agent, e.g. BDDE, in an aqueous solution, mixing until dissolution, and then exposing to cross-linking conditions, which may further comprise combining the resulting mixture of hyaluronic acid and a cross-linking agent, with an aqueous base, e.g. sodium hydroxide solution, and exposure to at least one of heating for 2-5 hours to a temperature between 40° C. and 50° C., preferably for about 3 hours at about 45° C., and keeping for 12-20 hours at between 22° C. and 27° C., preferably at about 25° C. for about 15 hours. A dried highly cross-linked hyaluronic acid powder may be produced by cross-linking hyaluronic acid with a suitable cross-linking agent as described for the cross-linked hyaluronic acid, preferably with the use of higher amount of cross-linking agent than needed for cross-linked hyaluronic acid, e.g. between 150% and 500% of the latter amount, to furnish a precursor gel to dried highly cross-linked hyaluronic acid powder. Thus, the cross-linking reagent may be present at different concentrations, at cross-linking conditions, e.g. between 0.8 and 5% wt, preferably between 0.9 to 3% wt, in the precursor gels for the preparation of the dried highly cross-linked hyaluronic acid powder.

The precursor gel may then be milled and dried. Preferably, the precursor gel is dried, e.g. using a lyophilizer, and then milled to a desired particle size. The milling (grinding) of the dried precursor gel may be accomplished by any technique known in the art, e.g. by a hammer mill, a cutting mill, a revolving high-impact mill, a ball mill; preferably, the milling is performed aseptically, such as in a tube mill. The ground particles may be fractionated according to their respective particle size, e.g. by sieving. Dried highly cross-linked hyaluronic acid may be provided in a form of a powder, e.g. as the plurality of particles. The average particle size particles may be of a size between 30 μm and 500 μm, preferably between 50 μm and 300 μm.

The gel components, e.g. the cross-linked hyaluronic acid and the precursor gel to the DHCL powder, may be milled, e.g. by extrusion, or by a high-shear mixer, to improve the flow properties during the further manufacturing steps. The milling may be performed in presence of additional liquid constituents, e.g. water and/or neutralization and/or osmolarity adjustment solution.

At any step of the manufacturing process, the mixture may be tested for quality assurance purposes. The applicable standard tests are known to a technically skilled person and include, e.g. rheometry, pH determination, residual cross-linking agent quantification, microscopy, centrifugation, and others.

The formulation may be filled into syringes and sterilized, e.g. by autoclaving, or by gamma irradiation. The sterile formulation may be used in a variety of applications, e.g. in tissue filling, such as tissue filling, e.g. wrinkle filling. The application of the compositions according to the present invention can be adjusted based on different ratios between the three components and their respective compositions. As a cosmetic product, the applications may range from body tissue filling, by using compositions with relatively high viscosity, to periorbital use, by using compositions with relatively low viscosity.

The terms “composite”, “composite gel”, “uniform gel”, “essentially homogeneous gel”, “composition” and the like, as used interchangeably herein, refer to the compositions of the present invention, with the choice of a particular term being dictated by emphasis given to a specific feature of the composition.

The invention is better understood in light of the appended examples, which should not be construed as limiting the invention in any aspect.

### EXAMPLES

Generally, unless indicated specifically otherwise, viscosity was measured with Brookfield viscometer using spindle LV-4, at 3 rpm, after 90 sec equilibration. Rheometry was performed using Malvern Kinexus Lab+ rheometer to determine the viscoelastic parameters, in parallel plates' configuration and controlled gap of 450 micrometers, with oscillatory tests at an amplitude determined to be in linear region by amplitude sweep test; frequency was changed between 10 and 10⁻³ Hz, at shearing strain of 0.5%, and the rheogram was recorded as elastic modulus (G′), viscous modulus (G″), and sometimes as the phase angle value (δ) of the complex modulus (G*).

The amounts of hyaluronic acid, as denoted herein, are given as supplied in the processes. The material contained certain degree of residual water, less than 15 weight percent (usually between 6.5 and 9 weight percent), thus the final concentration of hyaluronic acid is given where feasible.

### Example 1—Three Component Gel Composed of 89.55% CL-Gel-1, 9.95% NCLgel-1 and 0.5% of DHCL-1

Step 1: Preparation of DHCL-1 based on hyaluronic acid. Sodium hyaluronate (HA) of molecular weight 1.3-2.0 MDa (pharma grade) 6.54 g was added to 54.12 g water and 1.96 g of 1,4-Butanediol diglycidyl ether (BDDE), the mixture was mixed manually and then homogenized at 2000 rpm by Thinky planetary mixer. Thereafter, 11.23 g of 1M sodium hydroxide (NaOH) solution were added to the mixture, bringing to a total of 73.85 g. The mixture was than homogenized for 120 min at 300 rpm. The mixture was then placed in an oven set to 45° C. for 3 hours and sequentially 25° C. oven for additional 15 hours. The mixture was milled and then 70.59 g of the gel was neutralized by adding phosphate buffer at pH of about 7.3, and bringing to final pH with 1N HCl solution, total 103.29 g of neutralization solution, to give final pH of around 7. Final HA concentration was 3.3%.

The gel was dried from all liquids in lyophilizer, and then milled into powder with particles size smaller than 200 lam.

Step 2: Preparation of CL-Gel-1 Based on Hyaluronic Acid.

Sodium hyaluronate (HA) of molecular weight 1.3-2.0 MDa (pharma grade) 11.39 g was added to 94.30 g water and 1.14 g of 1,4-Butanediol diglycidyl ether (BDDE), the mixture was mixed for 30 min at 300 rpm. Thereafter, 19.57 g of 1 M sodium hydroxide (NaOH) solution were added to the mixture, bringing to a total weight of 126.4 g. The mixture was than homogenized for 120 min at 300 rpm. The mixture was then placed in an oven set to 45° C. for 3 hours and sequentially ° C. oven for additional 15 hours. The mixture was milled and then 120.9 g of the gel was neutralized by adding total of 379.1 g of neutralization solution as described above and mixing for 120 min at 300 rpm to give final pH of around 7. Final HA concentration was 2%.

Step 3: Preparation of NCL-Gel-1 Based on Hyaluronic Acid.

Sodium hyaluronate (HA) of molecular weight 1.3-2.0 MDa (pharma grade) 3.27 g was added to 146.73 g of phosphate buffer, the mixture was mixed for 120 min at 300 rpm to give cohesive gel. Final HA concentration was 2%.

Step 4: Preparation of the Final Bulk.

180 g of CL-gel-1 was added to 20 g of NCL-gel-1. Then 1 g of DHCL-1 was added to the mixture, the bulk was mixed manually and then homogenized at 2000 rpm by Thinky Mixer. The gel was finally degassed in vacuo, by subjecting it vacuum (˜30 mbar) for 30 minutes followed by milling and filling into 1.25-mL glass syringes. The syringes were sterilized by a steam autoclave at 121° C. for 20 minutes. A cohesive and viscoelastic gel was formed.

The final gel had pH value around 7. The gel was easily injectable through a needle: an injection force of 28 N was required for pushing the gel through a 25G/16 mm PIC needle, with a pushing rate of 1 mL/min. The gel had viscosity of 190 Pa*s, and G′ and G″, determined at 1 Hz at shear strain of 0.5%, were 93 Pa and 34 Pa, respectively.

### Example 2—Three Component Gel Composed of 49.75% CL-Gel-1, 49.75% NCL-Gel-1 and 0.5% of DHCL-1

Preparation of DHCL-1, CH-gel 1, and NCL-gel 1 are as described in example 1.

Step 4: Preparation of the Final Bulk.

100 g of CL-gel-1 was added to 100 g of NCL-gel-1. 1 g of DHCL-1 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

The final gel has pH value around 7. The gel was easily injectable through a needle: a force of 20 to 40 N was required for pushing the gel through a 25G/16 mm PIC needle, with a pushing rate of 1 mL/min.

### Example 3—Three Components Gel Composed of 89.1% CL-Gel-1, 9.9% NCL-Gel-1 and 1% of DHCL-1

Preparation of DHCL-1, CH-gel 1, and NCL-gel 1 are as described in example 1.

Step 4: Preparation of the Final Bulk.

13.50 g of CL-gel-1 was added to 1.50 g of NCL-gel-1. 0.15 g of DHCL-1 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

The final gel has pH value around 7. The gel was easily injectable through a needle: an injection force of 34 N was required for pushing the gel through a 25G/16 mm PIC needle, with a pushing rate of 1 mL/min. The gel had viscosity of 197 Pa*s, and G′ and G″, determined at 1 Hz at shear strain of 0.5%, were 125 Pa and 46 Pa, respectively.

### Example 4—Three Components Gel Composed of 74.6% CL-Gel-1, 24.9% NCL-Gel-1 and 0.5% of DHCL-1

Preparation of DHCL-1, CH-gel 1, and NCL-gel 1 are as described in example 1.

Step 4: Preparation of the Final Bulk.

15.00 g of CL-gel-1 was added to 5.00 g of NCL-gel-1. 0.10 g of DHCL-1 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

The final gel has pH value around 7. The gel was easily injectable through a needle: a force of 20 to 40 N was required for pushing the gel through a 25G/16 mm PIC needle, with a pushing rate of 1 mL/min.

### Example 5—Three Components Gel Composed of 89.55% CL-Gel-1, 9.95% NCL-Gel-1 and 0.5% of DHCL-2

Step 1: Preparation of a DHCL-2 based on hyaluronic acid. Sodium hyaluronate (HA) of molecular weight 1.3-2.0 MDa (pharma grade) 5.45 g was added to 45.10 g water and 1.36 g of 1,4-Butanediol diglycidyl ether (BDDE), the mixture was mixed manually and then homogenized at 2000 rpm. Thereafter, 9.36 g of 1M sodium hydroxide (NaOH) solution were added to the mixture, bringing to a total weight of 61.27 g. The mixture was than homogenized for 120 min at 300 rpm. The mixture was then placed in an oven set to 45° C. for 3 hours and sequentially 25° C. oven for additional 15 hours. The mixture was milled and then neutralized by adding 89.98 g of neutralization solution to give final pH of around 7. Final HA concentration was 3.4%. The gel was dried from all liquids and then milled into powder with particles size smaller than 200 μm.

Preparation of CH-gel 1 and NCL-gel 1 are as described in example 1.

Step 4: Preparation of the Final Bulk.

18.00 g of CL-gel-1 was added to 2.00 g of NCL-gel-1. 0.1 g of DHCL-2 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

The final gel has pH value around 7. The gel was easily injectable through a needle: a force of 26.8 N was required for pushing the gel through a 25G/16 mm PIC needle, with a pushing rate of 1 mL/min. The viscosity, determined as described in the methods section above, was 178.3 Pa*s.

The rheogram of the composition is shown in the FIG. 1. It can be readily observed that at the near-zero shear region (left side of the x-axis) that the viscous modulus G″ decreases sharply responsive to shearing, which is partly paralleled by the phase angle δ, indicating a significant increase in elasticity of the composition responsive to shearing, and the decrease in propensity to flow spontaneously and creep.

### Example 6—Three Components Gel Composed of 49.75% CL-Gel-1, 49.75% NCL-Gel-1 and 0.5% of DHCL-2

Preparation of DHCL-2, CH-gel 1, and NCL-gel 1 are as described in example 5.

Step 4: Preparation of the Final Bulk.

10.00 g of CL-gel-1 was added to 10.00 g of nNCL-gel-1. 0.10 g of DHCL-2 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed, with hyaluronic acid concentration of 2.3%.

The final gel has pH value around 7. The gel was easily injectable through a needle: the injection force was 12.7 Pa was required for pushing the gel through a 25G/16 mm PIC needle, with a pushing rate of 1 mL/min. The gel had viscosity 75.5 Pa*s, and G′ and G″, determined at 1 Hz at shearing strain of 0.5%, were 82 Pa and 46 Pa, respectively.

Further gels were prepared, comprising CL-gel-1, NCL-gel-1, and DHCL-2, in 49.5:49.5:1 ratio (Example 6b, 2.6% HA), and in 49:49:2 (Example 6c, 3.2% HA), as described above. The viscosity values were 141.3 and 196.8 Pa*s, respectively, for the gels of example 6b and 6c, their G′ and G″ were 128 and 64, and 145 and 76 Pa, and their injection force values were 15.2 N and 22.8 N.

### Example 7—Three Components Gel Composed of 74.6% CL-Gel-1, 24.9% NCL-Gel-1 and 0.5% of DHCL-2

Preparation of DHCL-2, CH-gel 1, and NCL-gel 1 are as described in example 5.

Step 4: Preparation of the Final Bulk.

15.00 g of CL-gel-1 was added to 5.00 g of NCL-gel-1. 0.10 g of DHCL-2 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed, with hyaluronic acid concentration of 2.3%.

The final gel has pH value around 7. The gel was easily injectable through a needle: the injection force was 17.8 Pa was required for pushing the gel through a 25G/16 mm PIC needle, with a pushing rate of 1 mL/min. The gel had viscosity 150.8 Pa*s.

Further gels were prepared, comprising CL-gel-1, NCL-gel-1, and DHCL-2, in 74.25:24.75:1 ratio (Example 7b, 2.6% HA), and in 73.5:24.5:2 (Example 7c, 3.2% HA), as described above. The viscosity value was 191.4 Pa*s for 7b and above 200 Pa*s for 7c and their injection force values were 19.7 N and 28.1 N, respectively.

### Example 8—Three Components Gel Composed of 89.0% CL-Gel-1, 10% NCL-Gel-1 and 1% of DHCL-2

Preparation of DHCL-2, CH-gel 1, and NCL-gel 1 are as described in example 5.

Step 4: Preparation of the Final Bulk.

13.53 g of CL-gel-1 was added to 1.53 g of NCL-gel-1. 0.15 g of DHCL-2 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

The final gel has pH value around 7. The gel was easily injectable through a needle: a force of 35.2±0.7 N (average and STD, n=3) was required for pushing the gel through a 27G/16 mm PIC needle, with a pushing rate of 1 mL/min. The viscosity, determined as described in the methods section above, was 197±0.1 Pa*s.

The rheogram of the composition is shown in the FIG. 2. Like in the Example 5, it can be readily observed that at the near-zero shear region (left side of the x-axis) that the viscous modulus G″ decreases sharply responsive to shearing, which is partly paralleled by the phase angle δ, indicating a significant increase in elasticity of the composition responsive to shearing, and the decrease in propensity to flow spontaneously and creep.

### Example 9—Three Components Gel Composed of ˜89% CL-Gel-1, ˜9% NCL-Gel-1 and 0.5%/1%/2% of Dry DHCL-2

Preparation of DHCL-2, CH-gel 1, and NCL-gel 1 are as described in example 5.

Step 4: Preparation of the Final Bulks (Three Hydrogels with 0.5%, 1% and 2% of DHCL-2 Components).

89.55 g of CL-gel-1 was added to 9.96 g of NCL-gel-1. 0.5 g of DHCL-2 was added to the mixture, the bulk was mixed manually and then homogenized at 2000 rpm. In a similar way, two other hydrogels were prepared to give final concentration of 1% and 2% for the DHCL-2 component. The gels were finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gels were formed.

The final gel has pH value around 7. The gel was easily injectable through a needle: a force of 20 to 40 N was required for pushing the gel through a 25G/16 mm PIC needle, with a pushing rate of 1 mL/min.

The rheological characteristics of the three gels were examined. The results indicate that increasing the DHCL-2 component % resulted in hydrogels with higher G′ (elastic modulus). G′ results at frequency of 1 Hz were: ˜100 (for gel with 0.5% DHCL-2), ˜150 (for gel with 1% DHCL-2) and ˜200 (for gel with 2% DHCL-2).

### Example 10—Three Components Gel Composed of 49.75% CL-Gel-2, 49.75% NCL-Gel-1 and 0.5% of DHCL-2

Step 1: Preparation of DHCL-2 as described in example 5.

Step 2: Preparation of CLG-2 Based on Hyaluronic Acid.

Sodium hyaluronate (HA) of molecular weight 1.3-2.0 MDa (pharma grade) 5.42 g was added to 95.67 g water and 1.09 g of 1,4-Butanediol diglycidyl ether (BDDE), the mixture was mixed for 30 min at 300 rpm. Thereafter, 18.72 g of 1 M sodium hydroxide (NaOH) solution were added to the mixture, bringing to a total weight of 120.9 g. The mixture was than homogenized for 120 min at 300 rpm. The mixture was placed in an oven set to 45° C. for 3 hours and sequentially 25° C. oven for additional 15 hours. The mixture was milled and then neutralized by adding 379.1 g of neutralization solution and mixing for 120 min at 300 rpm to give final pH of around 7. Final HA concentration was 1%.

Step 3: Preparation of NCL-gel-1 as described in example 1.

Step 4: Preparation of the Final Bulk.

10.00 g of CL-gel-2 was added to 10.00 g of NCL-gel-1.0.1 g of DHCL-2 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

### Example 11—Three Components Gel Composed of 49.75% CL-Gel-1, 49.75% NCL-Gel-2 and 0.5% of DHCL-2

Preparation of DHCL-2 and CH-gel 1, are as described in example 5.

Step 3: Preparation of NCL-Gel-2

Sodium hyaluronate (HA) of molecular weight 1.3-2.0 MDa (pharma grade) 1.64 g was added to 148.36 g of phosphate buffer, the mixture was mixed for 120 min at 300 rpm to give cohesive gel. Final HA concentration was 1%.

Step 4: Preparation of the Final Bulk.

10.00 g of CL-gel-1 was added to 10.00 g of NCL-gel-2. 0.10 g of DHCL-2 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

### Example 12—Three Components Gel Composed of 49.75% CL-Gel-1, 49.75% NCL-Gel-3 and 0.5% of DHCL-2

Preparation of DHCL-2 and CH-gel 1, are as described in example 5.

Step 3: Preparation of NCL-Gel-3

Sodium hyaluronate (HA) of molecular weight 1.3-2.0 MDa (pharma grade) 4.92 g was added to 145.08 g of phosphate buffer, the mixture was mixed for 120 min at 300 rpm to give cohesive gel. The final concentration of HA was 3%.

Step 4: Preparation of the Final Bulk.

10.00 g of CL-gel-1 was added to 10.00 g of NCL-gel-3. 0.10 g of DHCL-2 was added to the mixture, the bulk was mixed manually and then homogenized at 2000 rpm. The gel was finally degassed in vacuo, by subjecting it vacuum for 30 minutes followed by milling and filling into 1.25-mL glass syringes, which were sterilized by a steam autoclave at 121° C. for 20 minutes. A cohesive and viscoelastic gel was formed.

### Examples 13—Further Compositions with CL-Gel-1, NCL-Gel-1, and DHCL-2

Preparation of components was as described in the Example 6.

The final blending was performed to final ratios of CL-gel-1, NCL-gel-1, and DHCL-1, in 89.55:9.95:0.5 ratio (Example 13a, 2.3% HA), and in 90:9:1 (Example 13b, 2.6% HA). The viscosity values were 178.4 and 196.7 Pa*s, respectively, for the gels of example 13a and 13b, and their injection force values were 26.8 N and 31.4 N.

Further blends were performed to final ratios of CL-gel-1, NCL-gel-1, and DHCL-2, in 59.7:39.8:0.5 ratio (Example 13c, 2.3% HA), in 59.4:39.6:1 (Example 13d, 2.6% HA), in 79.6:19.9:0.5 ratio (Example 13e, 2.3% HA), in 79.2:19.8:1 (Example 13f, 2.6% HA), as described above. The viscosity values were 102.0, 135.9, 141.5 and 186.8 Pa*s, respectively, for the gels of example 13c-13f, their G′ and G″ were 69 and 40, 85 and 44, 71 and 32, and 87 and 40 Pa, respectively, and their injection force values were 12.1 N, 14.6 N, 19.3 N and 23.1 N.

### Examples 14—Further Compositions with NCL-Gel-4, CL-Gel-1, and DHCL-2

Preparation of components CL-gel-1, and DHCL-2 was as described for the Example 6.

NCL-gel 4 was prepared by dissolving sodium hyaluronate (HA) of molecular weight 1.3-2.0 MDa (pharma grade) 4.0 g was added to 146 g of phosphate buffer, the mixture was mixed for 120 min at 300 rpm to give cohesive gel. The final concentration of HA was 2.5%.

The final blending was performed, as described in the example 6, to final ratios of CL-gel-1, NCL-gel-4, and DHCL-1, in 89.55:9.95:0.5 ratio (Example 14a, 2.4% HA), in 74.625:24.845:0.5 (Example 14b, 2.5% HA), and in 49.75:49.75:0.5 (Example 14c, 2.6% HA). The viscosity values were 183.6, 161.8, and 175.9 Pa*s, respectively, for the gels of example 14a-14c, their G′ and G″ were 79 and 36, 84 and 43, and 117 and 85 Pa, respectively, and their injection force values were 32 N, 24.4 N and 22.3 N.

### Examples 15—Further Compositions with NCL-Gel-1 and DHCL-2, and Further CL-Gels

Preparation of components NCL-gel-1, and DHCL-2 was as described for the Example 6.

Step 2: Preparation of CLG-3 Based on Hyaluronic Acid.

Sodium hyaluronate (HA) of molecular weight 1.3-2.0 MDa (pharma grade) 5.42 g was added to 95.67 g water and 1.09 g of 1,4-Butanediol diglycidyl ether (BDDE), the mixture was mixed for 30 min at 300 rpm. Thereafter, 18.72 g of 1 M sodium hydroxide (NaOH) solution were added to the mixture, bringing to a total weight of 120.9 g. The mixture was than homogenized for 120 min at 300 rpm. The mixture was placed in an oven set to 45° C. for 3 hours and sequentially 25° C. oven for additional 15 hours. The mixture was milled and then neutralized by adding 224.3 g of neutralization solution and mixing for 120 min at 300 rpm to give final pH of around 7. Final HA concentration was 1.5%.

Step 2a: Preparation of CLG-4 Based on Hyaluronic Acid.

Sodium hyaluronate (HA) of molecular weight 1.3-2.0 MDa (pharma grade) 5.42 g was added to 95.67 g water and 1.09 g of 1,4-Butanediol diglycidyl ether (BDDE), the mixture was mixed for 30 min at 300 rpm. Thereafter, 18.72 g of 1 M sodium hydroxide (NaOH) solution were added to the mixture, bringing to a total weight of 120.9 g. The mixture was than homogenized for 120 min at 300 rpm. The mixture was placed in an oven set to 45° C. for 3 hours and sequentially 25° C. oven for additional 15 hours. The mixture was milled and then neutralized by adding 161.4 g of neutralization solution and mixing for 120 min at 300 rpm to give final pH of around 7. Final HA concentration was 1.8%.

The final blending was performed, as described in the example 6, to final ratios of CL-gel-2, NCL-gel-1, and DHCL-2, in 89.55:9.95:0.5 ratio (Example 15a, 1.4% HA). Further blends were prepared using CL-gel-3, NCL-gel-1, and DHCL-2, in a 89.55:9.95:0.5 ratio (Example 15b, 1.9% HA), and 74.625:24.845:0.5 (Example 15c, 2.0% HA). The viscosity values were 39.2, 107.9, and 85.9 Pa*s, respectively, for the gels of example 15a-15c, their G′ and G″ were 30 and 10, 40 and 17, and 46 and 22 Pa, respectively, and their injection force values were 9.7 N, 18.2 N and 13.7 N.

Further blends were prepared, as described in the example 6, to final ratios of CL-gel-4, NCL-gel-1, and DHCL-2 (particles between 50 and 100 microns), in 89.73:9.97:0.3 ratio (Example 15d, 2.0% HA), 89.55:9.95:0.5 ratio (Example 15e, 2.1% HA), and 94.525:4.975:0.5 ratio (Example 15f, 2.1% HA). The viscosity values were 131, 137.8, and 152.2 Pa*s, respectively, for the gels of example 15d-15f, their G′ and G″ were 50 and 25, 54 and 27, and 51 and 24 Pa, respectively, and their injection force values were 21.9 N, 23.5 N and 27.9 N.

### Comparative Example 1—Cross-Linked Gel of Hyaluronic Acid

Manufacturing a similar gel using the amounts of hyaluronic acid and the cross-linking agent as in the Example 5 resulted in a hard gel. Therefore a 2-% HA gel was prepared for comparison purposes.

Sodium hyaluronate (HA) of molecular weight 1.3-2.0 MDa (pharma grade) 10.87 g was added to 90.22 g water and 1.09 g of 1,4-Butanediol diglycidyl ether (BDDE), the mixture was mixed for 30 min at 300 rpm. Thereafter, 18.72 g of 1 M sodium hydroxide (NaOH) solution were added to the mixture, bringing to a total weight of 120.9 g. The mixture was than homogenized for 120 min at 300 rpm. The mixture was then placed in an oven set to 45° C. for 3 hours and sequentially ° C. oven for additional 15 hours. The mixture was milled and neutralized by adding 379.10 g of neutralization solution as described above and mixing for 120 min at 300 rpm to give final pH of around 7. Final HA concentration was 2%.

The rheogram of the composition is shown in the FIG. 3. It can be readily observed that at the almost throughout the shearing range, particularly at near-zero shear region (left side of the x-axis), the viscous modulus G″ increases continuously responsive to shearing, which is partly seconded by the phase angle δ. This indicates that the gel complies to the shearing and that it may creep even at low shear values.

### Example 16—Testing the Compositions in Tissue Filling Application

The compositions were prepared according to the Example 5. Upon approval of the product, it was administered to 6 patients, for wrinkle filling. The patients were assessed by the practitioners that administered the product, immediately after the application, several weeks after the administration, and several months after the administration (interim results, study ongoing). The assessment was done on subjective scoring scale from 0 to 5, with 0 being no improvement relative to no treatment, 5 being complete correction. Similarly, the practitioners were requested to summarize their experience with the Comparative Formulation, comprising only cross-linked hyaluronic acid gel, according to the same scale.

The evaluations are summarized in the Table below. The values are given as average±standard deviation.

| | Initial | | Short term | | | Long term | | |
|---|---|---|---|---|---|---|---|---|
| Treatment | n | scores | n | time | Score | n | time | Score |
| Example 5 | 6 | 4.83 ± | 6 | 2 weeks | 5.00 ± | 2 | 6 months | 5.00 ± |
| | | 0.41 | | | 0.00 | | | 0.00 |
| Comparative | 6 | 4.50 ± | 6 | 2 weeks | 4.17 ± | 2 | 6 months | 3.50 ± |
| | | 0.84 | | | 0.41 | | | 0.71 |

1. A process of manufacturing a hyaluronic acid composition, said process comprises combining free hyaluronic acid gel, cross-linked hyaluronic acid gel, and dried highly cross-linked hyaluronic acid gel, and mixing to obtain an essentially homogeneous gel which is homogeneous upon visual inspection versus ample light source and under magnification of up to ×3.

2. The process according to claim 1, wherein said free hyaluronic acid gel is provided by combining hyaluronic acid and an aqueous buffer solution, and mixing until dissolution.

3. The process according to claim 1, wherein said cross-linked hyaluronic acid gel is provided by combining in an aqueous medium hyaluronic acid or a salt thereof and a cross-linking agent, subjecting the resultant mixture to cross-linking conditions, and completing the cross-linking reaction.

4. The process according to claim 3, wherein said subjecting to cross-linking conditions comprises increasing the pH of the medium, and said completing the cross-linking reaction comprises allowing the reaction mixture to stand, and/or neutralizing said reaction mixture.

5. The process according to claim 1, wherein said dried highly cross-linked hyaluronic acid is provided by drying a precursor gel of highly cross-linked hyaluronic acid, and grinding it to particle size below 500 microns, preferably to below 250 microns.

6. The process according to claim 5, wherein said drying is effected by lyophilizing.

7. The process according to claim 5, wherein said precursor gel of highly cross-linked hyaluronic acid is provided by combining in an aqueous medium hyaluronic acid or a salt thereof and a cross-linking agent, subjecting the resultant mixture to cross-linking conditions, and completing the cross-linking reaction.

8. The process according to claim 7, wherein said subjecting to cross-linking conditions comprises increasing the pH of the medium, and said completing the cross-linking reaction comprises allowing the reaction mixture to stand, and/or neutralizing said reaction mixture.

9. The process according to claim 3, wherein said cross-linking agent is 1, 4-butanediol diglicydyl ether (BDDE).

10. The process according to claim 7, wherein an amount of said cross-linking agent in said precursor gel of highly cross-linked hyaluronic acid is between 150% and 500% higher than corresponding amount of said cross-linking agent in said cross-linked hyaluronic acid gel, on weight basis relative to a respective amount of hyaluronic acid.

11. The process according to claim 1, wherein an amount of said free hyaluronic acid gel is between 5 and 45 weight percent of the total weight of said essentially homogeneous gel, optionally between 7 and 25 weight percent.

12. The process according to claim 1, wherein an amount of said dried highly cross-linked hyaluronic acid gel is between 0.25 and 3.5 weight percent of the total weight of said essentially homogeneous gel.

13. The process according to claim 1, wherein an amount of said cross-linked hyaluronic acid gel is between 45 and 95 weight percent of the total weight of said essentially homogeneous gel, optionally between 70 and 95 weight percent.

14. The process according to claim 1, further comprising sterilizing said essentially homogeneous gel, optionally by autoclaving said essentially homogeneous gel.

15. The process according to claim 1, wherein said homogeneous gel is inseparable by centrifugation up to 120 minutes at 16,000 g-force.

16. The process according to claim 1, wherein said homogeneous gel is characterized in that that a plot of viscous modulus G′ versus frequency demonstrates a local minimum in near-zero region at frequencies between 0 and 5×10³ Hz, at frequency sweep test of said homogeneous composition.

17. A hyaluronic acid composition comprising water, free hyaluronic acid, cross-linked hyaluronic acid, and dense highly cross-linked hyaluronic acid, wherein said composition comprises between 0.5 and 9 weight percent of hyaluronic acid, wherein said composition is injectable, wherein said composition is an essentially homogeneous gel upon visual inspection versus ample light source and under magnification of up to ×3.

18. The composition according to claim 17, wherein said dense highly cross-linked hyaluronic acid is at least partially swollen particle of dried highly cross-linked hyaluronic acid gel.

19. The composition according to claim 17, wherein said cross-linked hyaluronic acid and said dense highly cross-linked hyaluronic acid comprise hyaluronic acid cross-linked with a cross-linking agent, wherein an amount of said cross-linking agent in said dense highly cross-linked hyaluronic acid is between 150% and 500% higher than the amount in said cross-linked hyaluronic acid, on weight basis relative to a respective amount of hyaluronic acid.

20. The composition according to claim 19, wherein said cross-linking agent is 1, 4-butanediol diglicydyl ether (BDDE).

21. The composition according to claim 17, wherein the amount of said free hyaluronic acid is between 5 and 45 weight percent of the total weight of said composition, optionally between 7 and 25 weight percent.

22. The composition according to claim 17, wherein the amount of said cross-linked hyaluronic acid is between 45 and 95 weight percent of the total weight of said composition, optionally between 70 and 95 weight percent.

23. The composition according to claim 17, wherein the amount of said dense highly cross-linked hyaluronic acid is between 0.25 weight percent and 3.5 weight percent.

24. The composition according to claim 17, which is inseparable by centrifugation for up to 120 minutes at 16,000 g-force.

25. The composition according to claim 17, wherein said composition is characterized in that that a plot of viscous modulus G versus frequency demonstrates a local minimum in near-zero region at frequencies between 0 and 5×10³ Hz, at frequency sweep test of said homogeneous composition.
